(19) United States
(12) Patent Application Publication
(10) Pub. No.: US 2007/0043008 A1
(43) Pub. Date: Feb. 22, 2007

(54) Condensed Bicyclo[3.1.0] Hex-4-EN Derivatives Useful as Antibacterial Agents

(76) Inventor: Eric Lattmann, Birmingham (GB)

Correspondence Address: FISH & RICHARDSON P.C., PO BOX 1022, MINNEAPOLIS, MN, 55440-1022, US

(21) Appl. No.: 10/546,269

(22) PCT Filed: Feb. 9, 2004
(86) PCT No.: PCT/GB04/00483

§ 371 Date: Nov. 3, 2006

(30) Foreign Application Priority Data
Feb. 20, 2003 (GB) 0303874.2

(51) Int. Cl. A61K 31/396; C07D 491/02

(52) U.S. Cl. 514/183; 548/961

(57) ABSTRACT

According to a first aspect of the present invention there is provided a compound of formula (I): where, A is carbon, sulphur, oxygen or nitrogen B is carbon or nitrogen, X is oxygen or sulphur, Y is hydrogen, a halogen, a substituted or unsubstituted heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties Z is hydrogen, a halogen, a substituted or unsubstituted heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl, and sulphur equivalents of said carbonyl and oxycarbonyl moieties, excluding the compound where A and X are oxygen, B is nitrogen, Y is chlorine and Z is methyl. The invention also relates to the use of the compounds to prevent or ameliorate bacterial infections.

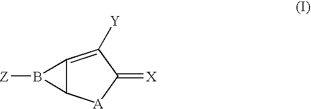

The present invention relates to novel compounds, said compounds exhibiting biological activity.

In an article [Lattmann et al; Pharm Pharm col Lett (2001) 1, 5-8] relating to the potential of furanones as anti-cancer agents 4-chloro-6-methyl-2-oxa-6-azabicyclo[3.1.0]hex-4-en-3-one was reported as a minor by-product which was obtained in 0.5% yield in the synthesis of a series of pseudoesters, pseudoanhydrides, pseudo-acid chlorides, pseudocarbamates and pseudoamines. As far as the inventors are aware, this is the only publication of a compound having a bicyclo[3.1.0]hex-4-ene structure and in all other respects, the 2-oxa-6-azabicyclo[3.1.0] hetero-cycle represents an entirely novel chemical class of compounds.

According to a first aspect of the present invention there is provided a compound of formula (I): 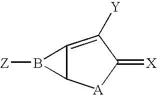
where,

A is carbon, sulphur, oxygen or nitrogen

B is carbon or nitrogen,

X is oxygen or sulphur,

Y is hydrogen, a halogen, a substituted or unsubstituted heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties Z is hydrogen, a halogen, a substituted or unsubstituted heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl, and sulphur equivalents of said carbonyl and oxycarbonyl moieties, excluding the compound where A and X are oxygen, B is nitrogen, Y is chlorine and Z is methyl.

The scope of the invention also extends to salts, particularly physiologically acceptable salts of the compounds of formula (I).

Preferably said alkyl-containing moieties (e.g. alkyl, alkyloxy etc.) are C₁-C₁₂, more preferably C₁-C₆ and most preferably C₁ to C₄.

Preferably said alkenyl and said alkynyl moieties are C₂-C₁₂, more preferably C₂-C₆ and most preferably C₂ to C₄.

Examples of suitable substituents for said heterocyclic, alkyl, alkenyl, alkynyl and aryl moieties include halo, amino, nitro, hydroxy and cyano moieties.

Preferably, said heterocyclic moiety is a monocyclic ring comprising at least one of oxygen, sulphur and nitrogen. More preferably said monocyclic ring is a 3 to 7 membered ring.

Preferably, said aryl moiety is substituted or unsubstituted phenyl.

Preferably, said cyclic alkyl moiety is a 3 to 7 membered ring and said cyclic alkenyl and alkynyl moieties are preferably, 4 to 7 membered rings.

Preferably, A is sulphur or oxygen and most preferably oxygen.

Preferably, B is nitrogen.

Preferably, X is oxygen.

Preferably, Y is hydrogen or a halogen. More preferably, Y is chlorine or bromine.

Preferably, Z is hydrogen, or any of the following which may be substituted or unsubstituted; C₁-C₄ alkyl, benzyl, aroylmethyl, allyl, or morpholinoethyl. Most preferably, Z is methyl, benzyl, substituted benzyl, ethyl, propyl, isopropyl or isobutyl.

Particularly preferred compounds of the first aspect are in accordance with formulae (II), (III), (IV) or (V):

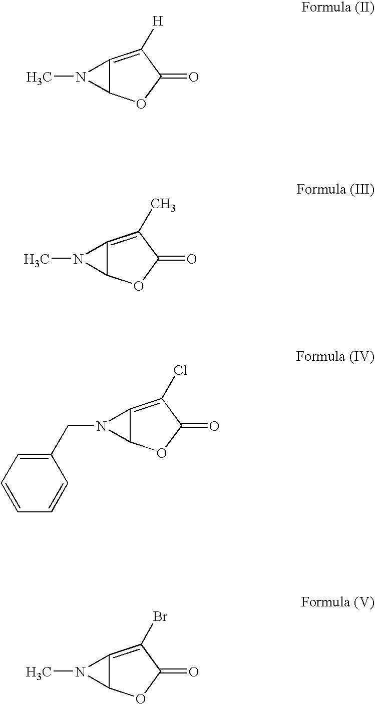

In a second aspect, the present invention relates to the use of a compound of formula (I) as a medicament. More specifically, but not exclusively, the second aspect relates to the use of a compound of formula (I) as a medicament for the treatment of a pathogenic infection, especially a bacterial infection. For the avoidance of doubt it is to be noted that the specific compound excluded from the first aspect is not excluded from the second aspect.

The present invention is based on the surprising discovery by the inventors that compounds of formula (I) exhibit broad spectrum antibacterial activity. Even more surprising is the discovery that in some cases activity is greater against antibiotic-resistant strains than the non-resistant equivalents.

The use of the compounds of the present invention as a medicament is particularly suited to ameliorate or prevent infections caused by Acinetobacter spp., Escherichia Coli, Enterobacter spp., Klebsiella pneumoniae, Pseudomonas aeruginosa, Staphylococcus aureus.

Particularly preferred compounds of the second aspect are in accordance with formulae (II), (III) (IV), (V) and (VI):—

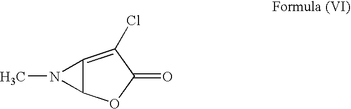

The second aspect of the present invention also resides in the use of a compound of formula (I) in the manufacture of a medicament for the treatment of a bacterial infection.

The invention further resides in a method of treating a human or non-human mammal afflicted with a bacterial infection, comprising administering to said mammal a therapeutically effective amount of a compound of formula (I).

Examples of uses in animal health include medicaments used in the production of shrimps/prawns in tanks as well as the mass production of chicken.

Administration may be by any known route e.g. by intravenous, intramuscular, or intrathecal (spinal) injection, intranasal, topical administration as an ointment, salve, cream or tincture, oral administration as a tablet, capsule (hard or soft; gelatin or non-gelatin based), suspension or liquid and nasal administration as a spray (e.g. aerosol).

Preferably, administration is oral or topical.

The compound of formula (I) is preferably in admixture with one or more excipients, carriers, emulsifiers, solvents, buffers, pH regulators, flavourings, colourings, preservatives, or other commonly used additives in the field of pharmaceuticals as appropriate for the mode of administration.

According to a third aspect of the invention there is provided a bactericidal composition comprising a compound of formula (I).

Compositions and medicaments containing the compound of formula (I) may also comprise one or more other active agents selected from, for example, antiviral, antifungal, antibacterial, and anti-inflammatory agents.

Embodiments of the invention will now be described by way of example only.

Referring to scheme 1, the mucohalogen acids such as mucochloric acid and mucobromic acid were reacted with simple commercially available formamides such as methylformamide, benzylformamide and formamide to give under reflux conditions in toluene the desired bicyclic furanone compounds (method A). Alternatively, the parent 4-halo-2-oxa-6-azabicyclo[3.1.0] was furnished by heating the mucohalogen acids with formamide under similar reaction conditions. This versatile oxa-6-aza-bicyclo[3.1.0] furanone building block was alkylated at 0° C. in THF with sodium hydride as a base using various alkylating agents to provide a series of alkylated bicyclic furanones (method B). Method A is the method of choice for simple and small formamides. Method B allows the introduction of a highly diverse range of substituents/functionalities on atom B as shown in formula (I) such as the nitrogen.

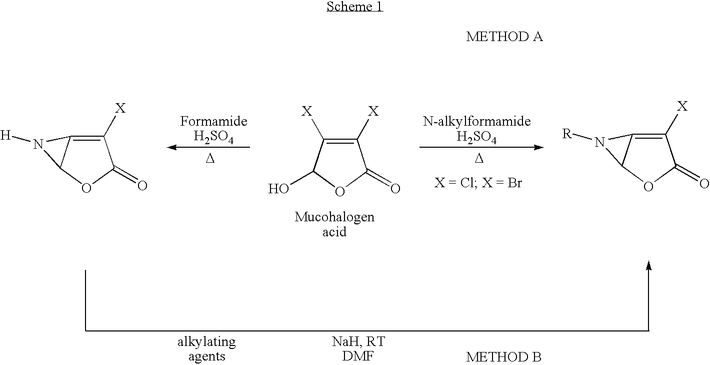

### EXAMPLE 1

### 4-chloro-6-methyl-2-oxa-6-azabicyclo[3.1.0]hex-4-en-3-one

Dry Mucochloric acid (15.0 g, 88.8 mmol) and N-methylformamide (9.46 g, 180 mmol) were refluxed in toluene (100 ml) with 1% of conc. H₂SO₄ using a Dean stark trap. After 55 hrs the mixture was cooled to room temperature and poured into 300 ml of water. The mixture was extracted 3 times with 100 ml of ether and the combined organic layers were dried with magnesium sulphate The solvents were removed in vacuum giving a viscous crude oil. This crude oil was transferred to the top of a column and was extracted with a mixture of petrol ether/ether 9:1. The target compound was obtained as white solid.

Yield: 65%. R_{f} (PE/ether)=0.83. Mol. Formula: C₅H₄ClNO₂. Mol.

Weight: 145.54. IR (KBr-disc) υ max: 2985, 2844, 1790, 1624, 1248, 1167, 954 cm⁻¹. MS (APCI+): 146, 148 (M+H) m/z. ¹H-NMR (CDCl₃) 300K δ: 3.07 (s, 3H, —CH₃), 6.65 (s, 1H, —CH) p.p.m.

¹³C-NMR (CDCl₃) 300K δ: 24.4 (N—CH₃), 126.8 (N—C—O), 140.9 (CCl), 164.9 (═C—N), 167.8 (C═O) p.p.m.

### EXAMPLE 2

### 4-bromo-6-methyl-2-oxa-6-azabicyclo[3.1.0]hex-4-en-3-one

Dry Mucobromic acid (20 g) and N-methylformamide (20 g, 4 eq) were refluxed in toluene (120 ml) with 1% of conc. H₂SO₄ using a Dean stark trap. After 72 hrs the mixture was cooled to room temperature and poured into 300 ml of water. The mixture was extracted 3 times with 100 ml of ether and the combined organic layers were dried with magnesium sulphate. The solvents were removed in vacuum giving a viscous crude oil. This crude oil was transferred to the top of a column and was extracted with a mixture of petrol ether/ether 9:1. The target compound was obtained as white solid.

Yield: 57%. IR (KBr-disc) υ max: 2988, 2863, 1790, 1623, 1248, 1167, 954 cm⁻¹. MS (APCI+): 191,193 (M+H) m/z. ¹H-NMR (CDCl₃) 300K δ: 3.09 (s, 3H, —CH₃), 6.75 (s, 1H, —CH) p.p.m.

¹³C-NMR (CDCl₃) 300K δ: 27.4 (N—CH₃), 132.2 (N—C—O), 140.9 (CBr), 162.6 (═C—N), 167.1 (C═O) p.p.m.

### EXAMPLE 3

### 4-chloro-2-oxa-6-azabicyclo[3.1.0]hex-4-en-3-one

Dry Mucochloric acid (15.0 g, 88.8 mmol) and N-formamide (7.5 g) were refluxed in toluene (100 ml) with 1% of conc. H₂SO₄ using a Dean stark trap. After 55 hrs the mixture was cooled to room temperature and poured into 300 ml of water. The mixture was extracted 3 times with 100 ml of ether and the combined organic layers were dried with magnesium sulphate. The solvents were removed in vacuum giving a viscous crude oil. This crude oil was transferred to the top of a column and was extracted with a mixture of petrol ether/ether 9:1. The target compound was obtained as white solid.

Yield: 42%. MS (APCI+): 131/133. IR (KBr-disc) υ max: 2981, 1788, 1624, 1248 cm⁻¹. ¹H-NMR (CDCl₃) 300K δ: 8.07 (s, 1H, NH), 6.67 (s, 1H, —CH) ppm.

### EXAMPLE 4

### 4-chloro-6-benzyl-2-oxa-6-azabicyclo[3.1.0]hex-4-en-3-one

Dry Mucochloric acid (8 g) and N-benzylformamide (7 g, 1.1 eq.) were refluxed in toluene (100 ml) with 1% of conc. H₂SO₄ using a Dean stark trap. After 55 hrs the mixture was cooled to room temperature and poured into 200 ml of a saturated solution of sodium hydrogen carbonate. The mixture was extracted 3 times with 100 ml of ether and the combined organic layers were dried with magnesium sulphate. The solvents were removed in vacuum giving a brown oil. This crude oil was transferred to the top of a column and was extracted with a mixture of petrol ether/ether 10:1. The target compound was obtained as white solid.

Yield: 25%. MS (APCI+): 221/223 (M+H) m/z. ¹H-NMR (CDCl₃) 300K δ: 7.4-7.2 (m, 5H, Ar); 6.2+5.9 (s, 1H, C5H); 5.35-5.15 (m, 2H, CH2) ppm.

Mass spectrometric analyses was obtained by Atmospheric Pressure Chemical Ionisation (APCI), negative or positive mode, using a Hewlett-Packard 5989b quadrupole instrument. This was connected to an electrospray 59987A unit with an automatic injection (Hewlett-Packard 1100 series autosampler). Samples were dissolved in HPLC grade methanol, toluene or acetonitrile. Both Proton and Carbon NMR spectra were obtained on a Brucker AC 250 instrument, operating at 250 MHz, calibrated with the solvent reference peak or TMS. IR spectra were plotted from KBr discs on a Mattson 300 FTIR Spectrophotometer.

Biological Activity Evaluation

The Minimal inhibitory concentration (MIC) and the Minimal bactericidal concentration (MBC) have been tested against various bacteria including patient isolates and antibiotic resistant strains such as S. aureus ATCC 25923, E. coli ATCC 25922, P. aeruginosa ATCC 27853.

Inoculum Preparation

The bacteria were streaked on a nutrient agar plate to obtain a freshly isolated colony subsequently incubated overnight at 37° C. 4-5 isolated colonies were added into Mueller Hinton broth (MHB) solution, incubated for 4 hrs at 37° C. The turbidity was adjusted to the McFarland tube and the solution was diluted with MHB to 1:200.

Antibacterial Dilution Tested

The test solution was diluted with dimethyl sulfoxide (DMSO) and MHB in the ratio of 1:4 to get a final concentration of 512 μg/ml. 50 μl of MHB were added to each of twelve wells except the first well. Dilutions were made, mixed and the solutions were then incubated overnight at 37° C. The MIC, the lowest concentration, which showed a clear solution, was examined for each Example compound.

In order to determine the MBC, a loopful of each clear well was streaked onto a nutrient containing agar plate, which was subsequently incubated overnight at 37° C. The MBC was then determined as the lowest concentration which has shown no visible colony for each chemical.

Results

The first step in the evaluation was to determine the zone of inhibition on agar plates comparing the Example compounds with ampicillin and chloramphenicol as standards. Following this initial screening, the MIC/MBC was determined as previously described and the results are outlined in Table 1 for selected examples.

TABLE 1
MIC and MBC.

| | MIC/MBC¹ (μg/ml) | | |
|---|---|---|---|
| Organism | Example 1 | Example 3 | Example 4 |
| Acinetobacter | A1 (8/16) | (32/32) | (4/8) |
| spp.² | A2 (64/64) | (32/64) | (8/16) |
| | A3 (16/32) | (64/128) | |
| | A4 (64/64) | (64/128) | |
| | A5 (32/32) | | |
| Escherichia coli² | E1 (32/32) | (64/128) | (16/16) |
| | E2 (64/64) | (128/128) | |
| | E3 (64/64) | (64/128) | |
| | E4 (32/64) | (128/128) | |
| | E5 (64/64) | | |
| Enterobacter | En1 (32/64) En3 | (64/64) | (8/16) |
| spp². | (32/32) En4 (64/ | (64/64) | (16/32) |
| | 128) En5 (64/ | (128/128) | |
| | 64) | | |
| Klebsiella | K1 (64/64) | (64/64) | |
| pneumoniae² | K2 (32/64) | (128/128) | |
| | K3 (32/32) | (128/128) | |
| | K4 (128/128) | | |
| | K5 (64/64) | | |
| Pseudomonas | P7 (32/64) | (128/128) | |
| aeruginosa² | P10 (64/64) | (128/128) | |
| | P12 (128/128) | (128/128) | |
| | P13 (32/64) | (128/128) | |
| | P14 (32/64) | | |
| | P16 (32/64) | | |
| Staphylococcus | S1 (128/>128) | (128/>128) | |
| aureus² | S2 (128/>128) | (128/>128) | |
| | S3 (64/128) | | |
| | S4 (64/>128) | | |
| E. coli ATCC | (16/32) | (128/128) | (8/16) |
| 25922³ | | | |
| P. aeruginosa | (32/32) | (128/128) | (16/32) |
| ATCC³ 27853 | | | |
| S. aureus ATCC | (16/64) | (128/>128) | (4/8) |
| 25923³ | | | |

¹The first value in bracket represents the MIC and the second is the MBC.
²Patient isolates:
³resistant strains

It is interesting to note that Example 1 had a lower MIC for the antibiotic resistant strains than the corresponding non-resistant isolates. The compounds of the present invention may therefore represent a useful new class of antibacterial agents, which may be acting by a new biological mechanism/novel mode of action. Example 4 in particular shows good broad spectrum activity.

For clinical use, the compounds may be administered systemically (e.g. intravenously) for serious systemic infections such as septicaemia. However, it is anticipated that one of the principle uses of the compounds will be topical administration for the treatment of local infections, or as part of a program to eliminate bacteria from a carrier prior to surgery, for example, to prevent dissemination of infection before it arises.

Most important is oral administration and the use in capsules and tablets. Due to a high solubility in organic solvents, DMSO and DMSO/water a good bioavailability is anticipated of these lipophilic molecules.

### MEDICAMENT EXAMPLE 1

4-chloro-6-methyl-2-oxa-6-azabicyclo[3.1.0]hex-4-en-3-one is mixed with paraffin wax, softisan [™], hydroxypropyl methyl cellulose, polyglyceryl-4-caprate and glycerine to give an ointment containing 2 wt % of the active agent.

Treatment Regime

The ointment is rubbed into the infected area 3 to 4 times daily until the infection is eliminated.

### MEDICAMENT EXAMPLE 2

4-methyl-6-methyl-2-oxa-6-azabicyclo[3.1.0]hex-4-en-3-one is mixed with an inert carrier liquid to give a 1% w/v of the active agent and dosed to a spray applicator.

Treatment Regime

The medicament is sprayed intranasally 3 to 4 times daily for five day to eliminate anterior nares carriage of S. aureus.

In Vivo Screening

No acute toxicity was observed in mice up to a dose of 500 mg/kg.

1. A compound of formula (I): 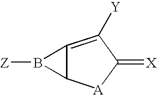
where,
A is carbon, sulphur, oxygen or nitrogen
B is carbon or nitrogen,
X is oxygen or sulphur,
Y is hydrogen, a halogen, a substituted or unsubstituted heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and
Z is hydrogen, a halogen, a substituted or unsubstituted heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl, and sulphur equivalents of said carbonyl and oxycarbonyl moieties,
or a physiologically acceptable salt thereof,
excluding the compound where A and X are oxygen, B is nitrogen, Y is chlorine and Z is methyl.

2. The compound of claim 1, wherein said alkyl-containing moieties are C₁-C₁₂ moieties.

3. The compound of claim 2, wherein said alkyl-containing moieties are C₁-C₆ moieties.

4. The compound of claim 1, wherein said alkenyl and said alkynyl moieties are C₂-C₁₂ moieties.

5. The compound of claim 4, wherein said alkenyl and said alkynyl moieties are C₂-C₆ moieties.

6. The compound of claim 1, wherein one or more of said substituted heterocyclic, alkyl, alkenyl, alkynyl and aryl moieties are substituted with halo, amino, nitro, hydroxy and cyano moieties.

7. The compound as claimed in claim 1, wherein said heterocyclic moiety is a monocyclic ring comprising at least one of oxygen, sulphur and nitrogen.

8. The compound as claimed in claim 7 wherein said monocyclic ring is a 3 to 7 membered ring.

9. The compound as claimed in claim 1, wherein said aryl moiety is substituted or unsubstituted phenyl.

10. The compound as claimed in claim 1, wherein said cyclic alkyl moiety is a 3 to 7 membered ring and said cyclic alkenyl and alkynyl moieties are 4 to 7 membered rings.

11. The compound as claimed in claim 1, wherein A is sulphur or oxygen.

12. The compound as claimed in claim 1, wherein B is nitrogen.

13. The compound as claimed in claim 1, wherein X is oxygen.

14. The compound as claimed in claim 1, wherein Y is hydrogen or a halogen.

15. The compound as claimed in claim 1, wherein Z is hydrogen, or any of the following which may be substituted or unsubstituted; C₁-C₄ alkyl, benzyl, aroylmethyl, allyl, or morpholinoethyl.

16. The compound as claimed in claim 1, wherein said compound has the formula (II), (III), (IV) or (V):

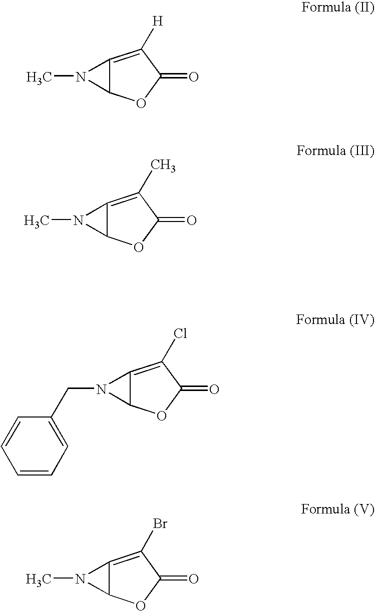

17. The use of a compound of formula (I), as defined in claim 1, as a medicament.

18. The use of claim 17, wherein said medicament is for the treatment of a pathogenic infection, especially a bacterial infection.

19. The use of claim 18 to ameliorate or prevent infections caused by Acinetobacter spp., Escherichia Coli, Enterobacter spp., Klebsiella pneumoniae, Pseudomonas aeruginosa, or Staphylococcus aureus.

20. The use of any one of claims 17 to 19, wherein said compound has the formula (II), (III) (IV) or (V), or (VI):—

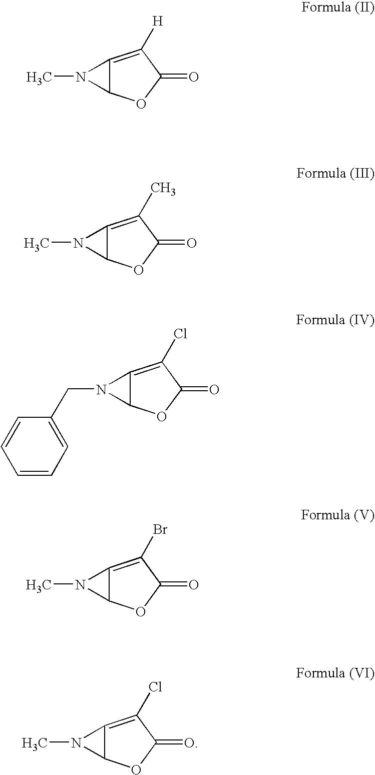

21. A method of treating a human or non-human mammal afflicted with a bacterial infection comprising administering to said mammal a therapeutically effective amount of a compound of formula (I), as defined in claim 1.

22. The method of claim 21, wherein said administration is oral or topical.

23. A bactericidal composition comprising a compound of formula (I), as defined in claim 1.

24. The compound of claim 14, wherein said halogen is chlorine or bromine.
